(19) United States
(12) Patent Application Publication
(10) Pub. No.: US 2023/0230259 A1
(43) Pub. Date: Jul. 20, 2023

(54) PHYSICAL ABILITY EVALUATION SERVER, PHYSICAL ABILITY EVALUATION SYSTEM, AND PHYSICAL ABILITY EVALUATION METHOD

(71) Applicant: HITACHI, LTD., Tokyo (JP)

(72) Inventors: Kenji Fujihira, Tokyo (JP); Masayoshi Ishibashi, Tokyo (JP)

(73) Assignee: HITACHI, LTD., Tokyo (JP)

(21) Appl. No.: 18/029,716

(22) PCT Filed: Dec. 18, 2020
(86) PCT No.: PCT/JP2020/047386

§ 371 Date: Mar. 31, 2023

(51) Int. Cl. G06T 7/246; G06T 7/00; G06T 7/215; G16H 50/30; G16H 30/40

(52) U.S. Cl. CPC G06T 7/251; G06T 7/0012; G06T 7/215; G16H 50/30; G16H 30/40; G06T 2207/30008; G06T 2207/30196; G06T 2207/10016

(57) ABSTRACT

A physical ability evaluation server includes an image processing unit that executes evaluation score calculation processing on a plurality of still images included in a measurement video to calculate a physical ability evaluation score, a physical ability evaluation unit that evaluates the physical ability based on the evaluation score, and an evaluation result notification unit that creates and outputs an evaluation report based on the evaluation result. The evaluation score calculation processing includes a first process of acquiring joint position coordinates by physique estimation for each still image, and a second process of acquiring physique information by segmentation for a first still image corresponding to a first target period, and a third process that calculates the evaluation score of physical ability by a predetermined calculation formula using the information acquired in the first and second processes with respect to a second still image corresponding to a second target period.

### TECHNICAL FIELD

The present invention relates to a physical ability evaluation server, a physical ability evaluation system, and a physical ability evaluation method and is preferably applied to a physical ability evaluation server, a physical ability evaluation system, and a physical ability evaluation method that automatically evaluate the physical ability of workers.

### BACKGROUND ART

As the working population declines, the increase in the workload on workers has become a serious social issue. In response to such situation, the government, companies, and related organizations are promoting work style reform efforts, and the human resource (HR) Tech market is expected to reach 250 billion yen in 2023. For companies to maintain or improve productivity as the birth rate declines and the population ages, it is important to invest in improving the productivity of middle-aged and older workers and extending working life. With the rapid spread of remote work due to COVID-19, there are concerns that workers will lose physical strength due to not commuting, and improving productivity and extending working life by improving the physical strength of workers has become important for the sustainability of corporate management.

Here, as means to evaluate the physical ability of workers, in addition to the method of evaluation by answering questionnaires to the subjects, there are known techniques for evaluating physical functions such as overhead squats that can evaluate flexibility of ankles and shoulder mobility reach that can evaluate flexibility of shoulders. As a measurement means used for evaluating physical ability, for example, wearable devices such as inertial sensors disclosed in PTL 1, physique estimation from camera images by deep learning AI disclosed in PTL 2, and segmentation for extracting a human area on an image disclosed in Non-PTL 1. There is also a method of combining a depth sensor and a neural network, as disclosed in PTL 3.

### CITATION LIST

### Patent Literature

PTL 1: JP2019-110990A
PTL 2: JP2017-080198A
PTL 3: JP2018-026131A

### Non-Patent Literature

Non-PTL 1: Xiaolong Liu, et al, “Recent progress in semantic image segmentation”, Artificial Intelligence Review, published online: 27 Jun. 2018

### SUMMARY OF INVENTION

### Technical Problem

However, the conventional physical ability evaluation methods described above have the following problems. First, when questionnaire responses are used, there is a problem with accuracy because the subject's responses may be arbitrary. Therefore, when evaluating physical ability, it was desirable to make a determination based on the movement and posture of the subject.

However, even when evaluating the physical ability based on the movement and posture of the subject, the automatic evaluation method using a wearable device as in PTL 1 requires an expensive device and requires 30 minutes or more per person to put on and take off sensors at all joint positions of the body, and thus, there was a problem that a large number of people could not be measured in a short time.

The method of estimating the physique from the image as in PTL 2 can acquire the joint position coordinates, but if the subject does not wear markers, the joint position coordinates are acquired by position estimation by deep learning AI, which makes highly accurate estimation difficult. Therefore, there was a problem that the method cannot be used for the determination which requires strictly measuring the human area, such as the degree of floating of the heel when squatting in an overhead squat, or the distance between both fists when both arms are wrapped around the back in a shoulder mobility reach. By using the segmentation technique disclosed in Non-PTL 1, it is possible to obtain the boundary of the human area from the image. However, since the boundary coordinates obtained from the above segmentation technique do not include information indicating the body position (heel, fist, and the like) to which the boundary coordinates belong, the technique cannot be used for physical ability evaluation.

The technique disclosed in PTL 3 can estimate changes in the hip rotation angle by inputting depth images and physique estimation results into a neural network. However, there is a problem that the technique cannot be applied to highly accurate determination based on the boundary coordinates of the human area.

The present invention has been made in consideration of the above points and is intended to propose a physical ability evaluation server, a physical ability evaluation system, and a physical ability evaluation method that enable automatic evaluation of physical ability based on highly accurate extraction of human areas while reducing costs in terms of time and price.

### Solution to Problem

To solve such a problem, the present invention provides a physical ability evaluation server that evaluates the physical ability of a subject based on a measurement video in which the subject performs a predetermined action required for evaluating the physical ability, the physical evaluation server including: an image processing unit that calculates an evaluation score of the physical ability by executing evaluation score calculation processing on a plurality of still images included in the measurement video; a physical ability evaluation unit that evaluates the physical ability based on the evaluation score calculated by the image processing unit; and an evaluation result notification unit that creates and outputs an evaluation report based on the evaluation result of the physical ability evaluation unit, in which the evaluation score calculation processing includes a first process of acquiring the joint position coordinates of the subject by physique estimation for each of the plurality of still images; a second process of acquiring human area coordinates forming a human area of the subject by segmentation for a first still image corresponding to a first target period among the plurality of still images, and acquiring predetermined physique information about the subject based on the human area coordinates; and a third process of calculating an evaluation score of the physical ability by a predetermined calculation formula using the information acquired in the first process and the second process for a second still image corresponding to a second target period different from the first target period among the plurality of still images.

To solve such a problem, the present invention provides a physical ability evaluation system that evaluates the physical ability of a subject based on a measurement video in which the subject performs a predetermined action required for evaluating the physical ability, the physical ability evaluation system including: a model action video player that plays a model action video for instructing the subject to execute the predetermined action; a measuring device that controls playing of the model action video by the model action video player and acquires the measurement video in which the subject has been photographed while playing the model action video; and a physical ability evaluation server that evaluates the physical ability of the subject based on the measurement video received from the measuring device, in which the physical ability evaluation server includes an image processing unit that calculates an evaluation score of the physical ability by executing evaluation score calculation processing on a plurality of still images included in the measurement video; a physical ability evaluation unit that evaluates the physical ability based on the evaluation score calculated by the image processing unit; and an evaluation result notification unit that creates and outputs an evaluation report based on the evaluation result of the physical ability evaluation unit, in which the evaluation score calculation processing includes a first process of acquiring the joint position coordinates of the subject by physique estimation for each of the plurality of still images; a second process of acquiring human area coordinates forming a human area of the subject by segmentation for a first still image corresponding to a first target period among the plurality of still images, and acquiring predetermined physique information about the subject based on the human area coordinates; and a third process of calculating an evaluation score of the physical ability by a predetermined calculation formula using the information acquired in the first process and the second process for a second still image corresponding to a second target period different from the first target period among the plurality of still images.

To solve such a problem, the present invention provides a physical ability evaluation method by a physical ability evaluation server that evaluates the physical ability of a subject based on a measurement video in which the subject performs a predetermined action required for evaluating the physical ability, the physical ability evaluation method including: an image processing step of calculating an evaluation score of the physical ability by executing evaluation score calculation processing on a plurality of still images included in the measurement video; a physical ability evaluation step of evaluating the physical ability based on the evaluation score calculated in the image processing step; and an evaluation result notification step of creating and outputting an evaluation report based on the evaluation result of the physical ability evaluation step, in which the evaluation score calculation processing in the image processing step includes a first process of acquiring the joint position coordinates of the subject by physique estimation for each of the plurality of still images; a second process of acquiring human area coordinates forming a human area of the subject by segmentation for a first still image corresponding to a first target period among the plurality of still images, and acquiring predetermined physique information about the subject based on the human area coordinates; and a third process of calculating an evaluation score of the physical ability by a predetermined calculation formula using the information acquired in the first process and the second process for a second still image corresponding to a second target period different from the first target period among the plurality of still images.

### Advantageous Effects of Invention

According to the present invention, it is possible to automatically evaluate physical abilities based on highly accurate extraction of a human area while reducing costs in terms of time and price.

### BRIEF DESCRIPTION OF DRAWINGS

FIG. 1 is a block diagram illustrating a configuration example of a physical ability evaluation system 1 according to an embodiment of the present invention.

FIG. 2 is a block diagram illustrating an example internal configuration of a physical ability evaluation server 10.

FIG. 3 is a sequence diagram illustrating an overall procedure example of physical ability evaluation by the physical ability evaluation system 1.

FIG. 4 is a flowchart illustrating a processing procedure of evaluation score calculation processing according to Example 1.

FIG. 5 is a flowchart illustrating a processing procedure of physical ability evaluation processing according to Example 1.

FIG. 6 is a diagram illustrating an example of a joint position estimation result table 121.

FIG. 7 is a diagram for explaining the use of human area coordinates in Example 1.

FIG. 8 is a diagram illustrating an example of an evaluation parameter management table 123.

FIG. 9 is an example of an image included in the model action video.

FIG. 10 is a diagram for explaining a method of calculating an inclination L representing the degree of floating of the heel.

FIG. 11 is a diagram illustrating an example of an evaluation score management table 122.

FIG. 12 is a diagram illustrating an example of an improvement training management table 124.

FIG. 13 is a diagram illustrating an example of an evaluation report.

FIG. 14 is a flowchart illustrating a processing procedure of evaluation score calculation processing according to Example 2.

FIG. 15 is a flowchart illustrating a processing procedure of physical ability evaluation processing according to Example 2.

FIG. 16 is a diagram illustrating an example of a joint position estimation result table 121.

FIG. 17 is a diagram for explaining the use of human area coordinates in Example 2.

FIG. 18 is a diagram for explaining a method of calculating the number of pixels M between fists.

FIG. 19 is a diagram illustrating an example of an evaluation score management table 122.

FIG. 20 is a diagram illustrating an example of an improvement training management table 124.

FIG. 21 is a diagram illustrating an example of an evaluation report.

### DESCRIPTION OF EMBODIMENTS

Hereinafter, embodiments of the present invention will be described in detail with reference to the drawings.

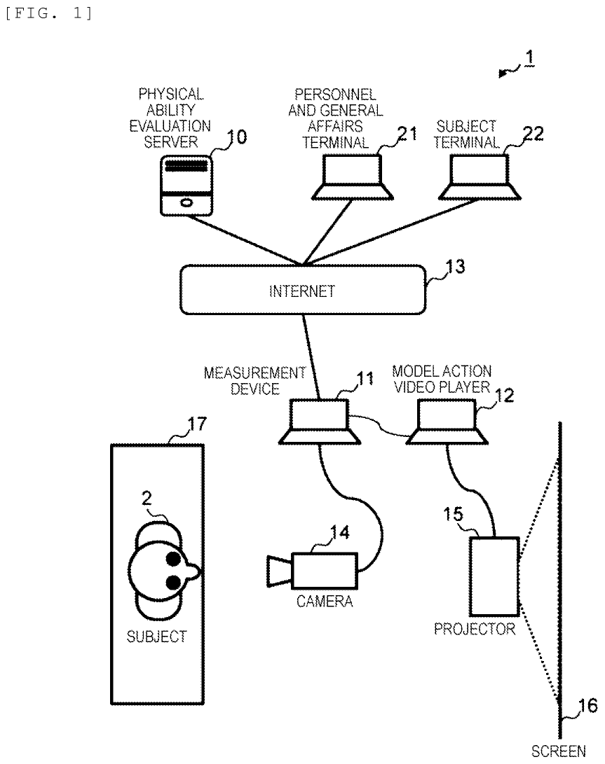

FIG. 1 is a block diagram illustrating a configuration example of a physical ability evaluation system 1 according to one embodiment of the present invention. The physical ability evaluation system 1 is a system that automatically evaluates the physical ability of a subject 2 (for example, a job seeker or a worker) based on measured data of the subject 2 and includes at least a physical ability evaluation server 10, a measuring device 11, and a model action video player 12.

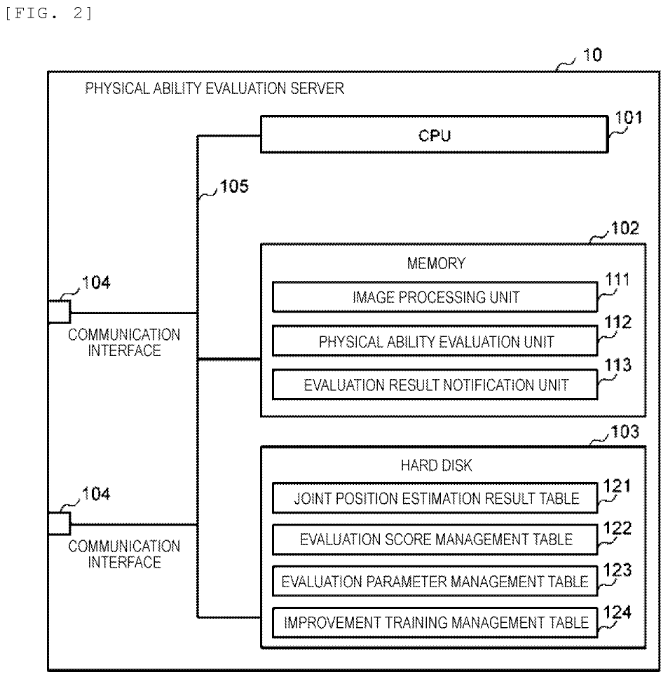

The physical ability evaluation server 10 is a computer that performs evaluation score calculation processing and physical ability evaluation processing, which will be described later, on the measurement video received from the measuring device 11 to evaluate the physical ability of the subject 2, and outputs the evaluation result. The physical ability evaluation server 10 is communicably connected to the measuring device 11 via a network (for example, the Internet 13). The internal configuration of the physical ability evaluation server 10 will be described later with reference to FIG. 2.

Note that the above network (Internet 13) is connected to a personnel and general affairs terminal 21 operated by a person in charge of personnel or a person in charge of general affairs, and a subject terminal 22 operated by the subject person 2, as an example of destinations for notification of evaluation results by the physical ability evaluation server 10. The personnel and general affairs terminal 21 and the subject terminal 22 are computers having a function of notifying the operator of the evaluation result (evaluation report described later) output from the physical ability evaluation server 10 by displaying, printing, or the like, and is, for example, a terminal such as a personal computer (PC) or a tablet.

The measuring device 11 is a device operated by a measurer who measures the physical information of the subject 2, and is, for example, a computer such as a notebook PC. In the present embodiment, as illustrated in FIG. 1, a camera 14 capable of capturing videos is connected to the measuring device 11 as an example of measuring means included in the measuring device 11. The model action video player 12 is also connected to the measuring device 11. When the physical information is measured, the measuring device 11 instructs the model action video player 12 to play the model action video representing the model action in the physical information measurement and instructs the measuring means (camera 14) to take a video of the subject 2.

When measuring physical information, the camera 14 takes a video of the subject 2 at a predetermined shooting position (for example, on a yoga mat 17) according to the instruction of the measuring device 11, and inputs the measurement video, which is the captured data, to the measuring device 11. Upon receiving the measurement video from the camera 14, the measuring device 11 transmits the measurement video to the physical ability evaluation server 10 after completing the measurement of the physical information (or in real time).

The model action video player 12 is a device capable of playing back video data prepared in advance and plays the model action video for physical information measurement prepared in advance on a predetermined display means according to the instructions of the measuring device 11. The model action video is a video that instructs the subject to execute a predetermined action necessary for the physical ability evaluation server 10 to evaluate the physical ability and includes a video that urges the subject (subject 2) to take a posture (for example, standing upright) from which physique information can be acquired and a video that urges specific postures (for example, squatting in an overhead squat, wrapping around the back with arms, and the like) required for evaluation of physical performance. In the present embodiment, as illustrated in FIG. 1, a projector 15 is shown as an example of display means connected to the model action video player 12. When the model action video is played by the model action video player 12, the projector 15 projects the model action video onto a screen 16 installed at a position visible to the subject 2.

FIG. 2 is a block diagram illustrating an example internal configuration of the physical ability evaluation server 10. As shown in FIG. 2, the physical ability evaluation server 10 includes a CPU 101, a memory 102, a hard disk 103, and a communication interface 104 as computer hardware configurations, and such configurations are connected to each other via a bus 105.

The central processing unit (CPU) 101 is an example of a processor included in the computer of the physical ability evaluation server 10. The memory 102 is a main storage device in the computer and stores programs and data.

The hard disk (hard disk drive (HDD)) 103 is an example of an auxiliary storage device in the computer of the physical ability evaluation server 10 and stores data referred to when executing programs stored in the memory 102 and data input from the outside, and the like. The auxiliary storage device in the physical ability evaluation server 10 is not limited to a hard disk and may be a solid state drive (SSD) or other flash memory, or a storage device externally connected to the computer.

The communication interface 104 is an interface for the computer of the physical ability evaluation server 10 to communicate with the outside, and data transmission and reception between the measuring device 11 and the personnel and general affairs terminal 21 are achieved by connecting to the Internet 13.

In FIG. 2, functional units such as an image processing unit 111, a physical ability evaluation unit 112, and an evaluation result notification unit 113 are shown in the memory 102. Such functional units are implemented by the CPU 101 executing a program stored in the memory 102 while referring to data stored in the hard disk 103 or the like as necessary.

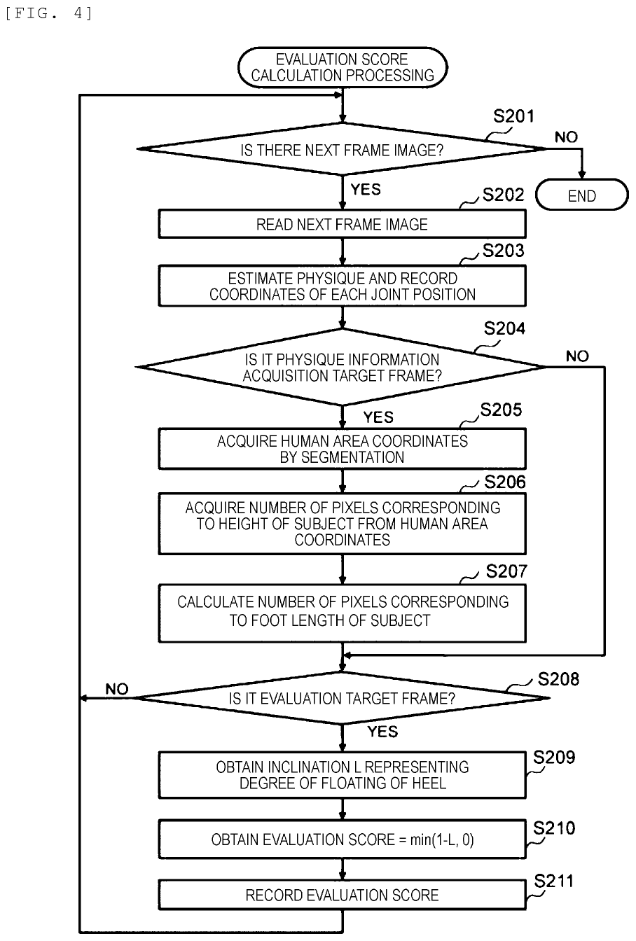

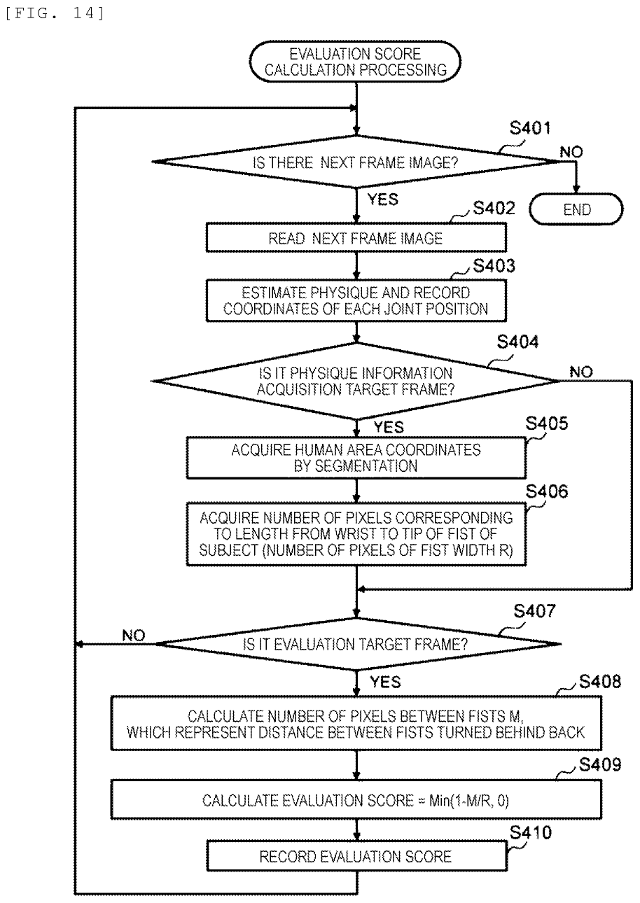

The image processing unit 111 has a function of executing evaluation score calculation processing on the measurement video received from the measuring device 11. Although the details will be described later with reference to FIGS. 4 and 14, the evaluation score calculation processing is processing performed on a still image of each frame in the measurement video during the processing target period, in which joint position coordinates of the subject 2 are recorded by the physique estimation, the human area coordinates forming the human area of the subject 2 are acquired by segmentation, and the evaluation score is calculated from the still image of each frame (evaluation target frame) in a predetermined evaluation target period and recorded.

Here, the processing target period and the evaluation target period in the measurement video will be added. The measurement video is recorded based on the playing state of the model action video replayed when the physical information is measured. In the measurement of physical information, it is assumed that a certain amount of delay time will occur when the subject 2 who has watched the model action video performs a specified posture or action. Therefore, when determining the action of the subject 2 in the measurement video based on the playing status of the model action video, it is preferable to determine the action at the timing added with the above-mentioned delay time. Therefore, in the present embodiment, for example, the timing obtained by adding the delay time to the play start timing of the model action video is set as the start timing of the processing target period in the measurement video. The end timing of the processing target period may be the timing obtained by adding the delay time to the play end timing of the model action video (which may be a predetermined timing before the play end). The evaluation target period in the measurement video is the period during which the evaluation score is calculated in the evaluation score calculation processing and corresponds to the period during which the subject 2 is in a specific evaluation target posture within the processing target period. In the present embodiment, different evaluation target postures can be determined depending on the evaluation items of the physical ability evaluation, so the evaluation target period also varies depending on the evaluation items of the physical ability evaluation. Specifically, for example, in Example 1, which will be described later, the evaluation target period is when the subject 2 is squatting in an overhead squat, and in Example 2, the evaluation target period is when the subject 2 has both arms wrapped around the back in a shoulder mobility reach.

Although details will be described later in each example, in the present embodiment, whether the posture is an evaluation target posture is determined by the posture of the subject 2 represented by the joint position coordinates obtained by physique estimation of the still image of the measurement video. To improve the accuracy of determination, the evaluation parameter management table 123 in which a predetermined playing position (play timing) for prompting the posture to be evaluated in the model action video is set can also be used for the above determination. The reason is because in the measurement of physical information, the subject 2 performs the action according to the instruction of the model action video, and thus, it is possible to predict a period during which the subject 2 can take the evaluation target posture in the measurement video by setting a predetermined playback position in the model action video.

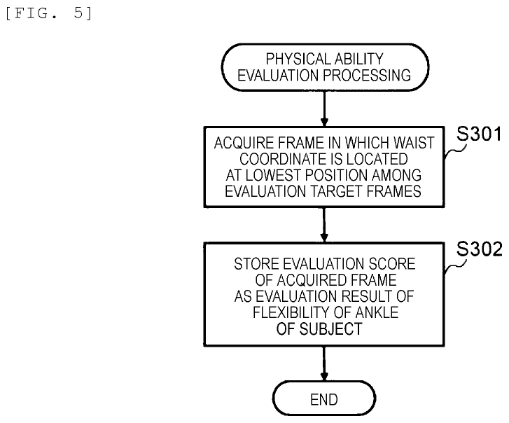

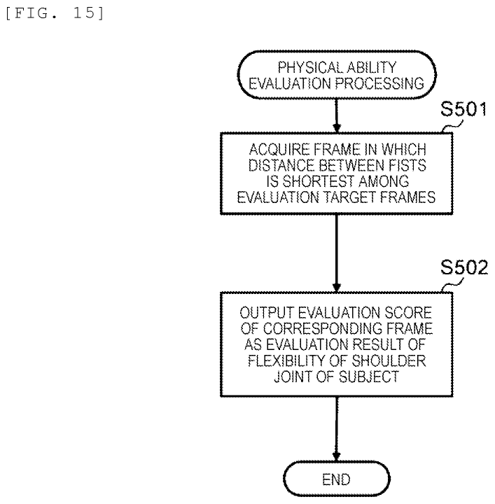

The physical ability evaluation unit 112 has a function of evaluating the physical ability of the subject 2 by executing the physical ability evaluation processing detailed in FIGS. 5 and 15 by using the evaluation score calculated by the image processing unit 111.

The evaluation result notification unit 113 has a function of creating an evaluation report including improvement training to be proposed to the subject 2 based on the evaluation result by the physical ability evaluation unit 112 and the evaluation result and notifying to the personnel and general affairs terminal 21 or the subject terminal 22 or the like with a predetermined output method. In the present embodiment, screen display via the Web is adopted as an example of an output method of the evaluation report, but the output method is not limited to the output method and may be an output method such as printing, e-mail, or the like.

FIG. 2 illustrates a joint position estimation result table 121, an evaluation score management table 122, an evaluation parameter management table 123, and an improvement training management table 124 as examples of data stored in the hard disk 103. A specific example of the data will be shown in the examples described later.

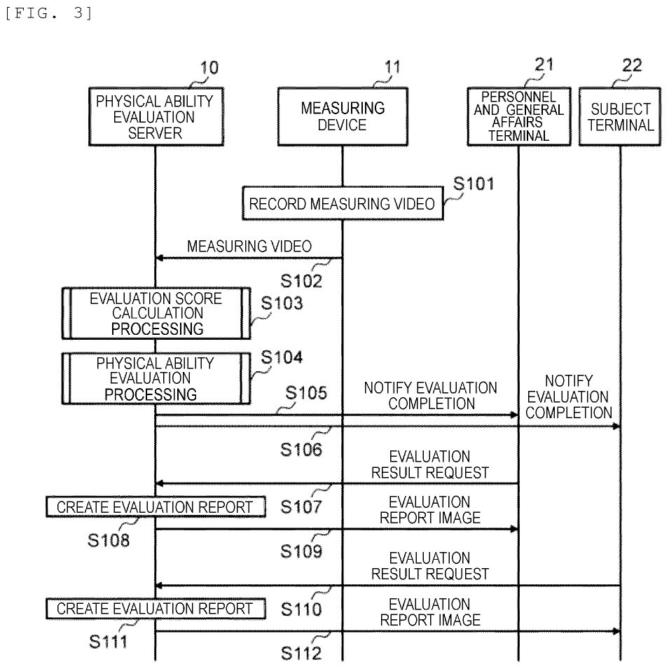

FIG. 3 is a sequence diagram illustrating an example of the overall procedure for physical ability evaluation by the physical ability evaluation system 1.

According to FIG. 3, first, the measuring device 11 measures the physical information of the subject 2. Specifically, the measuring device 11 records a measurement video of the subject 2 by taking a video of the subject 2 with the camera 14 while playing the model action video from the model action video player 12 (step S101). When the physical information is measured, the subject 2 is required to perform a predetermined posture and an action according to the model action video. Therefore, the measuring apparatus 11 records the measurement video based on the playing state of the model action video. Then, the measuring device 11 transmits the measurement video recorded in step S101 to the physical ability evaluation server 10 (step S102).

Next, in the physical ability evaluation server 10 that has received the measurement video in step S102, the image processing unit 111 executes evaluation score calculation processing on the measurement video, and calculates an evaluation score for each frame in the evaluation target period (step S103).

Next, in the physical ability evaluation server 10, the physical ability evaluation unit 112 executes physical ability evaluation processing using the evaluation score calculated in step S103 and evaluates the physical ability of the subject 2 (step S104). The evaluation result of step S104 is stored in the physical ability evaluation server 10.

Then, the physical ability evaluation server 10 (for example, the evaluation result notification unit 113) notifies the personnel and general affairs terminal 21 and the subject terminal 22 via the Internet 13 that the physical ability evaluation of the subject 2 has been completed (steps S105 and S106).

When the person in charge of human resources or the person in charge of general affairs operates the personnel and general affairs terminal 21 to request the provision of the evaluation result (step S107) after receiving the notification of step S105, the evaluation result notification unit 113 of the physical ability evaluation server 10 prepares an evaluation report based on the evaluation results obtained in step S104 (step S108), and displays the evaluation report on, for example, a website via the Internet 13, thereby providing the evaluation report to the terminal 21 (step S109).

When receiving the notification of step S106, when the subject 2 operates the subject terminal 22 to request the provision of the evaluation result (step S110), similarly to steps S108 and S109, the evaluation result notification unit 113 of the physical ability evaluation server 10 creates an evaluation report (step S111) and provides the evaluation report to the requesting subject terminal 22 via the Internet 13 (step S112).

The detailed processing procedures of the evaluation score calculation processing in step S103 and the physical ability evaluation processing in step S104 will be described for each embodiment described later with reference to separate drawings.

The above is the configuration and overall processing of the physical ability evaluation system 1 according to the present embodiment. Below, Example 1 and Example 2 are described as specific examples of physical ability evaluation by the physical ability evaluation system 1. Since Examples 1 and 2 are based on the above description of the physical ability evaluation system 1, the description of the configuration and processing described above will be omitted.

### Example 1

In Example 1, as an example of physical ability evaluation, a case will be described in which the flexibility of the ankle is evaluated by the degree of floating of the heel when squatting in an overhead squat. Insufficient ankle flexibility is known to be one of the main causes of low back pain, and the physical ability evaluation system 1 (physical ability evaluation server 10) can evaluate the degree of risk of low back pain of the subject 2 and propose improvement training for the subject 2 to prevent or eliminate low back pain by performing the physical ability evaluation of Example 1.

FIG. 4 is a flowchart illustrating the processing procedure of evaluation score calculation processing in Example 1, and FIG. 5 is a flowchart illustrating the processing procedure of physical ability evaluation processing in Example 1. As described above, the evaluation score calculation processing is executed by the image processing unit 111 and the physical ability evaluation processing is executed by the physical ability evaluation unit 112.

First, the evaluation score calculation processing in Example 1 will be described in detail with reference to FIG. 4. The evaluation score calculation processing is executed after the physical ability evaluation server 10 receives the measurement video from the measuring device 11, and the image processing unit 111 executes the processing shown in FIG. 4 on the still image of each frame included in the measurement video in the processing target period, in order from the frame corresponding to the start timing of the model action video (the timing to which a predetermined delay time is added to the start timing of the model action video).

According to FIG. 4, the image processing unit 111 first determines whether there is a next frame image included in the processing target period (step S201), and if there is a next frame image (Yes in step S201), the image processing unit 111 proceeds to step S202, and the next frame still image is read (step S202). Immediately after the start, it is naturally determined as YES in step S201. On the other hand, after the processing from step S202 on the image of the final frame of the processing target period is completed, it is determined in step S201 that the next frame image does not exist (NO in step S201), and the evaluation score calculation processing is ended.

When a still image (hereinafter referred to as a target image) is read in step S202, the image processing unit 111 performs physique estimation from the target image, acquires each joint position coordinate of the subject 2, and records the acquired coordinates in the joint position estimation result table 121 (step S203). As a method of estimating a physique from an image, an existing method may be used, for example, the method disclosed in PTL 2 can be used.

FIG. 6 is a diagram illustrating an example of the joint position estimation result table 121. A joint position estimation result table 210 shown in FIG. 6 is an example of the joint position estimation result table 121 in Example 1, and includes a subject ID 211, a frame 212, a waist x-coordinate 213, a waist y-coordinate 214, a knee x-coordinate 215, a knee y-coordinate 216, an ankle x-coordinate 217, and an ankle y-coordinate 218. The subject ID 211 is an identifier (ID) assigned to each subject 2 who is the subject of physical information measurement. The frame 212 indicates the frame number of the target image from which the joint position coordinates are acquired. Coordinate values representing the joint positions of the predetermined parts (waist, knee, and ankle) estimated in the physique estimation are recorded in the waist x-coordinate 213 to the ankle y-coordinate 218. Note that the data items of the joint position estimation result table 210 in FIG. 6 are merely an example, and in reality, the joint positions of more parts may be recorded.

Next, after step S203 is completed, the image processing unit 111 determines whether the current target image frame is the physique information acquisition target frame (step S204). The physique information acquisition target frame is a frame corresponding to a period during which the physique information of the subject 2 is acquired in a normal state, and different frames can be defined depending on the measurement contents of the physical information. In Example 1, the timing at which the subject 2 stands upright and stretches out is set as the physique information acquisition target frame. As a specific method for determining such a physique information acquisition target frame, for example, when the nose of the subject 2 is in the highest position compared to the target image of the previous frame, it is conceivable to be determined as a physique information acquisition target frame. The result of physique estimation in step S203 (that is, the position coordinates of the nose or joints near the nose) can be used to determine the position of the nose of the subject 2. As a device for narrowing down the comparison period, it may be limited to within several seconds after the video of the upright state is played in the model action video. If determining in step S204 that the frame is a physical information acquisition target frame (YES in step S204), the image processing unit 111 proceeds to step S205, and if determining not to be a physical information acquisition target frame (step S204 NO), the image processing unit 111 proceeds to step S208.

In step S205, the image processing unit 111 performs segmentation for extracting the human area from the image, and acquires the human area coordinates of the subject 2 from the image of the physique information acquisition target frame (the target image read in step S202). Next, the image processing unit 111 acquires the number of pixels corresponding to the height of the subject 2 from the human area coordinates acquired in step S205 (step S206), and further, based on the number of pixels corresponding to the height, the number of pixels corresponding to the foot length (sole length) of the subject 2 is converted (step S207).

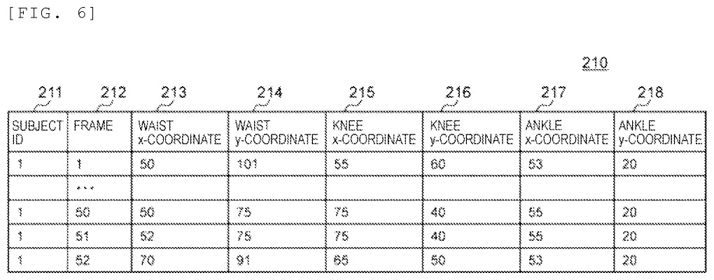

FIG. 7 is a diagram for explaining the use of human area coordinates in Example 1. The human area extraction result 220 shown in FIG. 7 is an example of the human area extracted by the segmentation in step S205 in Example 1 and shows the whole-body area of the subject 2 standing upright. The image processing unit 111 can obtain the number of pixels 221 corresponding to the height of the subject 2 by calculating the number of pixels in the vertical width of the human area extraction result 220 in step S206.

After the end of step S207, the image processing unit 111 determines whether the frame of the current target image is a frame (evaluation target frame) corresponding to the evaluation target period (step S208).

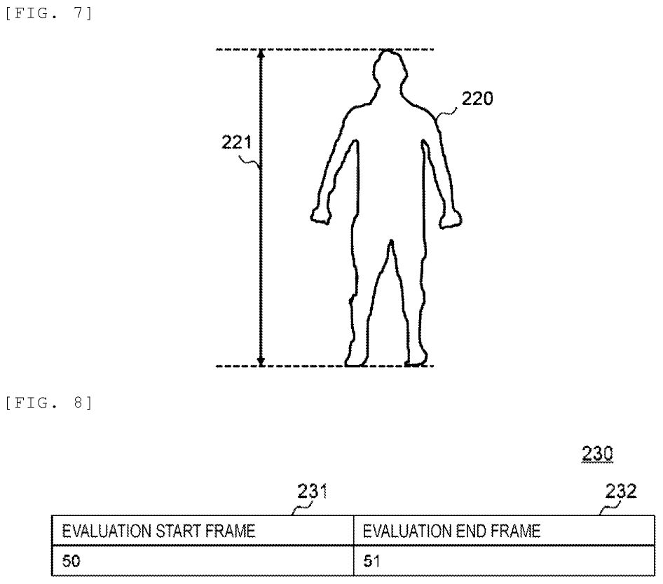

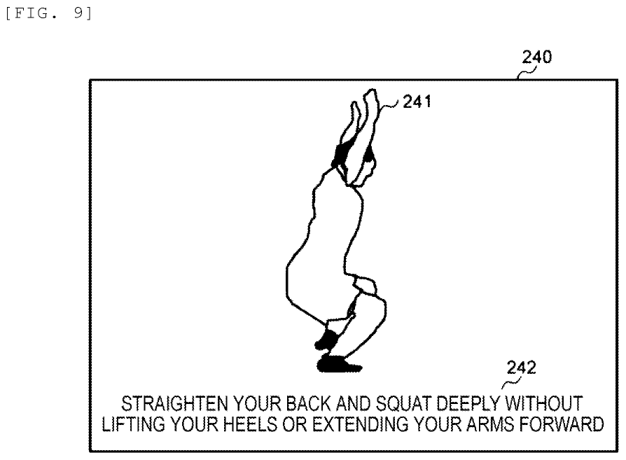

As described additionally in the description of the image processing unit 111 described above, the evaluation target period is a period during which the evaluation score is calculated in the evaluation score calculation processing and means a period during which the subject 2 is in a specific evaluation target posture. Specifically, in Example 1, since the posture in which the subject 2 squats down in an overhead squat is the posture to be evaluated, if the current target image takes such an evaluation target posture, it is determined as an evaluation target frame in step S208. Whether the posture of the subject 2 in the current target image is an evaluation target posture is determined based on the posture of the subject 2 indicated by the joint position coordinates obtained in the physique estimation in step S203. To improve the accuracy of determination, the evaluation parameter management table 123 in which a predetermined playing position (play timing) for prompting the posture to be evaluated in the model action video is set can also be used for determination. Details will be described later with reference to FIGS. 8 and 9.

If determining in step S208 that the frame is an evaluation target frame (YES in step S208), the image processing unit 111 proceeds to step S209. On the other hand, if determining in step S208 that the frame is not an evaluation target frame (NO in step S208), the image processing unit 111 ends the processing for the target image of the current frame, and moves to step S201 to proceed to the processing for the next frame image.

FIG. 8 is a diagram illustrating an example of the evaluation parameter management table 123. The evaluation parameter management table 230 shown in FIG. 8 is an example of the evaluation parameter management table 123 in Example 1 and is configured to include an evaluation start frame 231 and an evaluation end frame 232. In the evaluation start frame 231 and the evaluation end frame 232, the frame number at the start or end of the playing position (play timing) prompting the posture to be evaluated in the model action video is set, respectively. By adjusting the correspondence relationship between the frame numbers in the model action video and the frame numbers in the measurement video with an assumption of the delay time, the playing content in the model action video and the motion and posture of the subject 2 in the measurement video can be associated with each other.

Specifically, in the case of the evaluation parameter management table 230 of FIG. 8, the evaluation start frame 231 is the “50th” frame, and the evaluation end frame 232 is the “51st” frame. This means that the period between the 50th frame and the 51st frame is the timing at which the subject 2 is assumed to take the evaluation target posture. Therefore, in step S208, when the target image read in step S202 is the 50th or 51st frame image, the image processing unit 111 evaluates whether the target posture is actually taken based on the physique estimation result (joint position coordinates), and thus, it is possible to determine whether the current frame is the evaluation target frame.

A method for determining the posture to be evaluated based on the physique estimation results (joint position coordinates) will be described with reference to FIG. 9. FIG. 9 is an example of an image included in the model action video. The model action image 240 shown in FIG. 9 is an image illustrating one scene of the model action video played back by the model action video player 12 when the physical information is measured in Example 1 and is an image requesting the posture to be evaluated. More specifically, the model action image 240 includes a human image 241 squatting by overhead squat, and instruction comment 242 stating “Straighten your back and squat deeply without lifting your heels or extending your arms forward” to properly instruct the squat. The subject 2 imitates the model action image 240 and squats down. Therefore, the image processing unit 111 determines whether the posture of the subject 2 in the target image is the same as the human image 241 of the model action image 240, thereby enabling to determine whether the subject 2 is taking the evaluation target posture. As a specific method for determining the posture to be evaluated, for example, the image processing unit 111 uses the joint position coordinates recorded in step S203 to determine whether the posture is a squatting posture, that is, the posture to be evaluated when the waist height of the subject 2 is below a certain position (for example, 80% or less of the waist height in the case of standing upright).

In step S209, the image processing unit 111 calculates the inclination L representing the degree of floating of the heel by a predetermined calculation method using the processing results of steps S203 to S207 for the target image, which is the image of the evaluation target frame.

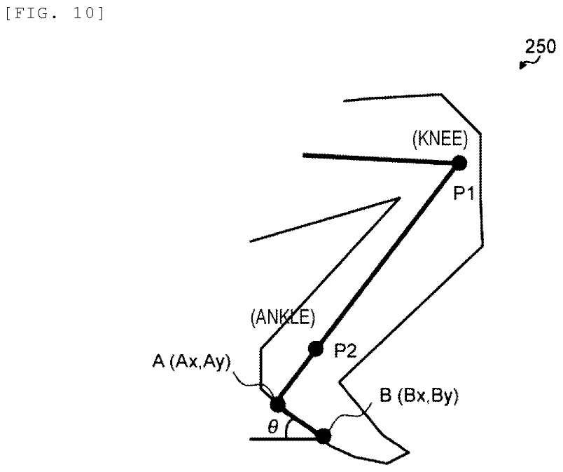

FIG. 10 is a diagram for explaining a method of calculating the inclination L, which represents the degree of floating of the heel. FIG. 10 shows an image 250 below the knee when the subject 2 is squatting during squats in the image of the evaluation target frame. A specific method of calculating the inclination L in step S209 will be described with reference to the image 250 of FIG. 10.

In calculating the inclination L, the image processing unit 111 first acquires the knee joint coordinates P1 and the ankle joint coordinates P2 using the physique estimation result (for example, the joint position estimation result table 210 in FIG. 6). When the line connecting P1 and P2 is extended in the direction of the ankle, the coordinates (Ax, Ay) of the point A that intersects with the boundary line of the human area (for example, the human area extraction result 220 in FIG. 7) are calculated.

Next, the image processing unit 111 calculates the coordinates (Bx, By) of the point B, which is moved from the point A by “foot length x coefficient (for example, 0.5)” along the boundary of the sole. The value of the coefficient may be any number between 0 and 1, but empirically around 0.5 is preferable.

Finally, the image processing unit 111 calculates the inclination of the angle (evaluation target angle θ) formed by the point A with respect to the point B in the horizontal direction as the inclination L representing the degree of floating of the heel. That is, the image processing unit 111 calculates the slope L by calculating “(Ay−By)/(Ax−Bx)” using the coordinates of the points A and B.

Returning to the description of FIG. 4, after calculating the inclination L representing the degree of floating of the heel in step S209, the image processing unit 111 calculates an evaluation score (step S210). The evaluation score is calculated based on the inclination L calculated in step S209. Specifically, “1-L” and “0” are compared, and the smaller value is used as the evaluation score.

Then, the image processing unit 111 records the evaluation score calculated in step S210 in the evaluation score management table 122 (step S211) and returns to step S201 for processing the next frame image.

FIG. 11 is a diagram illustrating an example of the evaluation score management table 122. The evaluation score management table 260 shown in FIG. 11 is an example of the evaluation score management table 122 in Example 1 and is table data configured with items of subject ID 261, frame 262, and evaluation score 263. In the evaluation score management table 260, the ID of the subject 2 is recorded in the subject ID 261, the frame number of the target image is recorded in the frame 262, and the evaluation score calculated in step S210 is recorded in the evaluation score 263.

Note that since the evaluation score is calculated only for the evaluation target frame image, the evaluation scores for the 50th and 51st frames are recorded in the present example. Specifically, according to the evaluation score management table 122 of FIG. 11, it is recorded that in the measurement video of the subject 2 to which the ID “1” is assigned, the evaluation score of the 50th frame image was “0.2” and the evaluation score of the 51st frame image was “0.3”.

As described above, by performing the processing of steps S201 to S211 in FIG. 4, the image processing unit 111 can calculate the evaluation score from a still image of the evaluation target frame included in the measurement video received from the measuring device 11 and record the evaluation score in the evaluation score management table 122.

Next, the physical ability evaluation processing in Example 1 will be described in detail with reference to FIG. 5. The physical ability evaluation processing is executed by the physical ability evaluation unit 112 with respect to the still image of each frame included in the measurement video received from the measuring device 11 by the measurement of the physical information of the subject 2, after the image processing unit 111 finishes the evaluation score calculation processing shown in FIG. 4.

According to FIG. 5, referring to the joint position estimation result table 210 shown in FIG. 6, the physical ability evaluation unit 112 first retrieves the record in which the waist coordinates (hip y 214) is located at the lowest position from the records having the frame 212 corresponding to the evaluation target frame and obtains the frame 212 of the record (step S301). In the present example, the evaluation target frames are the 50th frame and the 51st frame. When comparing the frames with the waist y-coordinate 214 in the joint position estimation result table 210, the waist position in the 51st frame, which is 73 (cm), can be seen as the lowest frame.

Next, the physical ability evaluation unit 112 refers to the evaluation score management table 260 illustrated in FIG. 11, acquires the evaluation score of the frame obtained in step 301, and stores the obtained evaluation score in a predetermined storage unit (for example, hard disk 103) as the evaluation result of flexibility of the ankle of the subject 2 (ankle flexibility evaluation result) (step S302). Specifically, since the frame acquired in step S301 is “51”, referring to the evaluation score management table 260, the evaluation score “0.3” of the 51st frame is stored as the ankle flexibility evaluation result.

As described above, by performing the processing of steps S301 and S302 in FIG. 5, the physical ability evaluation unit 112 can evaluate the evaluation score calculated from the measurement video by the evaluation score calculation processing and determine the physical ability evaluation result (specifically, the evaluation result of flexibility of the ankle of the subject 2).

Then, as described in FIG. 3, after the physical ability evaluation processing is completed, the evaluation result notification unit 113 notifies the completion of the evaluation (steps S105 and S106), and when the provision of the evaluation result is requested by the personnel and general affairs terminal 21 or the subject terminal 22 (steps S107 and S110), the evaluation result notification unit 113 creates an evaluation report (steps S108 and S111) and provides the report to the requester (steps S109 and S112).

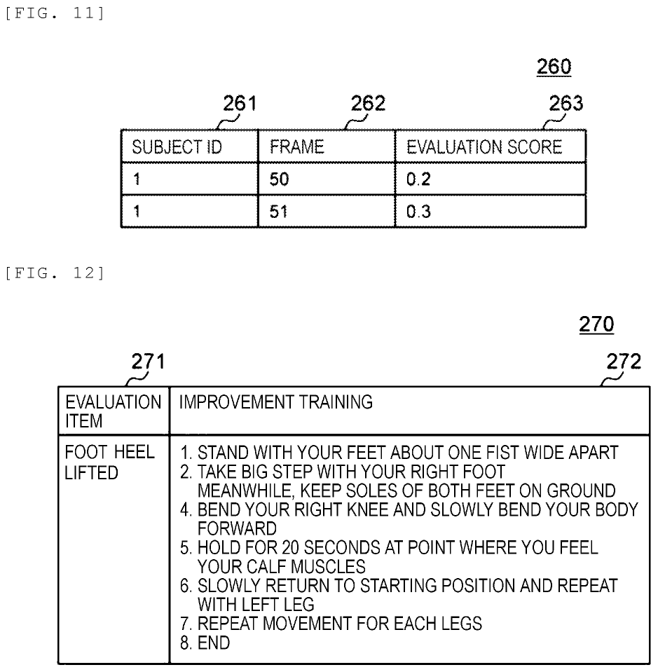

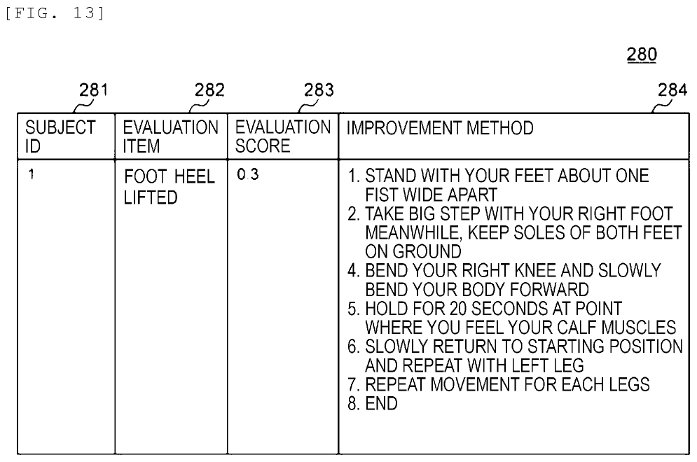

Here, a method of creating an evaluation report by the evaluation result notification unit 113 in Example 1 will be described. The evaluation result notification unit 113 uses the ankle flexibility evaluation result (evaluation score) of the subject 2 obtained by the physical ability evaluation processing and the improvement training management table 124 (see FIG. 12) stored in the hard disk 103 in advance to create an evaluation report (see FIG. 13) for physical ability evaluation of the subject 2.

FIG. 12 is a diagram illustrating an example of the improvement training management table 124. The improvement training management table 270 shown in FIG. 12 is an example of the improvement training management table 124 in Example 1 and is table data configured to include evaluation items 271 and improvement training 272 items. The evaluation item 271 describes evaluation items for physical ability evaluation. In the present example, since the flexibility of the ankle is evaluated based on the degree of lift of the heel when the subject squats down in an overhead squat, the evaluation item 271 is described as “foot heel lifted”. The improvement training 272 describes a training method recommended for improving the physical ability related to the evaluation item 271.

FIG. 13 is a diagram illustrating an example of an evaluation report. The evaluation report 280 shown in FIG. 13 is an example of an evaluation report regarding physical ability evaluation of ankle flexibility of the subject 2, and is table data configured to include subject ID 281, evaluation item 282, evaluation score 283, and improvement method 284.

In the evaluation report 280, the subject ID 281 indicates the ID assigned to the subject 2 and corresponds to the subject ID 211 of the joint position estimation result table 210 and the subject ID 261 of the evaluation score management table 260. The evaluation item 282 indicates the evaluation item of physical ability evaluation and corresponds to the evaluation item 271 of the improvement training management table 270.

The evaluation score 283 indicates an evaluation score representing the evaluation result of the physical ability evaluation specified from the subject ID 211 and the evaluation item 282. That is, in the present example, the evaluation score 283 describes the evaluation score of the ankle flexibility evaluation result (“0.3” according to the specific example described in step S302 of FIG. 5) obtained by the physical ability evaluation processing.

The improvement method 284 indicates a recommended training method for improving the physical ability related to the evaluation item 282. The evaluation result notification unit 113 can determine what kind of training method is described in the improvement method 284 based on the value of the evaluation score 283. For example, when the value of the evaluation score 283 is equal to or less than a predetermined reference value (for example, “0.5”), it is determined that improvement training needs to be proposed, and the described contents of the corresponding improvement training 272 from the improvement training management table 270 is described in improvement method 284. In the case of FIG. 13, since the evaluation score 283 is “0.3” which is equal to or less than the reference value, it is determined that improvement training needs to be proposed, and the training method described in the improvement training 272 of the improvement training management table 270 is recorded in the improvement method 284. On the other hand, if the value of the evaluation score 283 exceeds the reference value, it is determined that there is no need to propose improvement training, and a statement such as “improvement not required” is made. Note that the improvement method 284 is not limited to the above-described two-step determination of necessary and unnecessary, and the evaluation result notification unit 113 may make more diverse determinations and describe corresponding improvement methods.

As described above, the evaluation result notification unit 113 can create an evaluation report 280 including the evaluation result of the physical ability (ankle flexibility) evaluated from the measurement result of the subject 2 and the improvement training method based on the evaluation result and can provide the personnel and general affairs terminal 21 and the subject terminal 22 with the created evaluation report 280.

As described above, in Example 1, the physical ability evaluation system 1 (physical ability evaluation server 10) can evaluate the physical ability using an inexpensive device such as the camera 14 capable of capturing videos instead of an expensive device such as a wearable device and based on the captured measurement video of the movement and posture of the subject 2 without the need for time-consuming preparation such as attachment of the sensor. Therefore, it is possible to reduce the time and cost required for physical ability evaluation.

In the physical ability evaluation system 1 (physical ability evaluation server 10) according to Example 1, based on the play timing (playing position) of the model action video played by the model action video player 12, by managing the frames of the actions and postures performed by the subject 2 in the measurement video, the timing to acquire the physique information of the subject 2 (physical information acquisition target frame) and the timing to evaluate physical ability (evaluation target frame) in the measurement video can be specified. The physical ability evaluation system 1 (physical ability evaluation server 10) acquires the physical information of the subject 2 by physique estimation, acquires the human area coordinates by segmentation, and uses the acquisition results, thereby it is possible to acquire the feature amount (inclination L representing the degree of floating of the heel) of the evaluation target with high accuracy. Since the physical ability evaluation system 1 (physical ability evaluation server 10) evaluates the physical ability based on the feature amount of the evaluation target acquired as described above, the automatic evaluation of the physical ability can be performed based on the highly accurate extraction of the human area.

In Example 1, the physical ability evaluation system 1 (physical ability evaluation server 10) can provide not only the result of the automatic evaluation of physical ability but also the improvement method based on the result by presenting the evaluation report. Therefore, the person in charge of human resources can optimize the assigned department of the target person 2, and the subject 2 can learn the training for improving their own physical ability. Thus, according to the physical ability evaluation system 1 (physical ability evaluation server 10), by optimizing assigned departments and proposing improvement training based on the physical work ability evaluation results, productivity improvement and working life extension of middle-aged and older workers can be achieved.

### Example 2

In Example 2, as an example of physical ability evaluation, a case will be described in which the flexibility of the shoulder joint is evaluated based on the distance between both fists when behind the back is wrapped around by both arms in shoulder mobility reach. Insufficient flexibility of the shoulder is known as one of the main causes of shoulder stiffness. By performing the physical ability evaluation of Example 2, the physical ability evaluation system 1 (physical ability evaluation server 10) can evaluate the degree of risk of stiff shoulders of the subject to and propose improvement training for the subject 2 to prevent or eliminate stiff shoulders.

Note that in the description of Example 2, the description of parts common or similar to the description of Example 1 may be omitted or simplified.

FIG. 14 is a flowchart illustrating the processing procedure of evaluation score calculation processing in Example 2, and FIG. 15 is a flowchart illustrating the processing procedure of physical ability evaluation processing in Example 2. As described above, the evaluation score calculation processing is performed by the image processing unit 111 and the physical ability evaluation processing is performed by the physical ability evaluation unit 112.

First, the evaluation score calculation processing in Example 2 will be described with reference to FIG. 14. As in Example 1, the evaluation score calculation processing is executed after the physical ability evaluation server 10 receives the measurement video from the measuring device 11, and the image processing unit 111 executes the processing shown in FIG. 14 on the still images of each frame included in the measurement video of the processing target period in order from the frame corresponding to the start timing of the model action video (frame of the timing in which a predetermine delay time is added to the start timing of the model action video).

The processing from steps S401 to S405 in FIG. 14 are the same as the processing from steps S201 to S204 in FIG. 4 described in Example 1. For example, in step S403, the image processing unit 111 performs physique estimation from the target image read in step S402, acquires each joint position coordinate of the subject 2, and records the coordinates in the joint position estimation result table 121.

FIG. 16 is a diagram illustrating an example of the joint position estimation result table 121. A joint position estimation result table 310 shown in FIG. 16 is an example of the joint position estimation result table 121 in Example 2, and a table data configured to include items of a subject ID 311, a frame 312, a right wrist x-coordinate 313, a right wrist y-coordinate 314, a left wrist x-coordinate 315, and a left wrist y-coordinate 316. The subject ID 311 is an identifier (ID) assigned to each subject 2 who is a person to be measured for physical information. The frame 312 indicates the frame number of the target image from which the joint position coordinates are acquired. Then, in the right wrist x-coordinate 313 to the left wrist y-coordinate 316, coordinate values representing joint positions of predetermined parts (right wrist and left wrist) estimated by physique estimation are recorded.

After the processing of step S405, the image processing unit 111 obtains the physique information by obtaining the number of pixels corresponding to the length from the wrist to the tip of the first of the subject 2 (number of first width pixels R) from the human area coordinates obtained in step S405 (step S406).

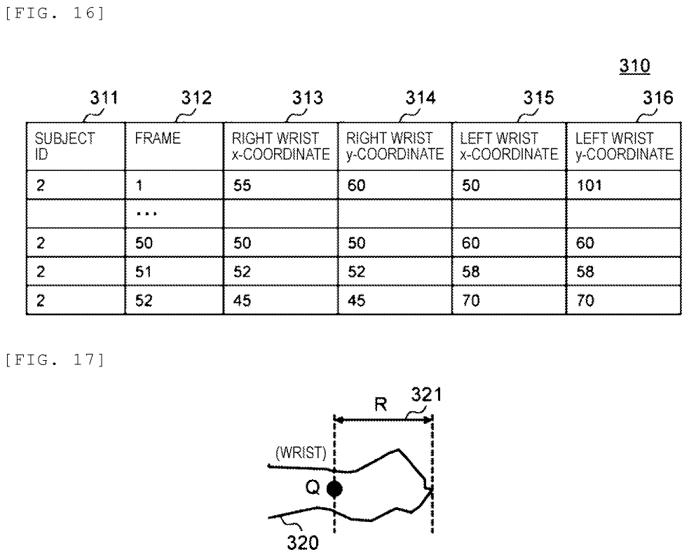

FIG. 17 is a diagram for explaining the use of human area coordinates in Example 2. A human area extraction result 320 shown in FIG. 17 is a partial example of the human area extracted by the segmentation in step S405 in Example 2 and shows the area beyond the wrist of the subject 2. A point Q is the joint position of the wrist acquired in step S403. In step S406, the image processing unit 111 can obtain the number of pixels of first width R by calculating the number of pixels 321 in the horizontal direction from the point Q of the human area extraction result 320 to the tip of the fist.

After step S406, the image processing unit 111 determines whether the frame of the current target image is the evaluation target frame (step S407), and if determining that it is an evaluation target frame (YES in step S407), the image processing unit 111 proceeds to step S408. If determining that the frame is not an evaluation target frame (NO in step S407), the image processing unit 111 ends the process for the target image of the current frame, and proceeds to step S401 to proceed to the processing for the image of the next frame.

As supplemented in the description of the image processing unit 111 described above, the evaluation target period is a period during which the evaluation score is calculated in the evaluation score calculation processing and means a period during which the subject 2 is in a specific evaluation target posture. Specifically, in Example 2, since the posture to be evaluated is the posture of the subject 2 in which both arms are wrapped around the back in shoulder mobility reach, in step S208, it is determined as an evaluation target frame when such an evaluation target posture is taken in the current targe image. As in the description of Example 1, whether the posture of the subject 2 in the current target image is the posture to be evaluated is determined based on the posture of the subject 2 indicated by the joint position coordinates obtained in the physique estimation in step S403. To improve the accuracy of determination, an evaluation parameter management table 123 in which a predetermined playing position (play timing) for prompting the posture to be evaluated in the model action video is set can also be used for determination. The details of the determination can be considered in the same manner as in Example 1, and thus, the description thereof will be omitted.

In step S408, the image processing unit 111 calculates the number of pixels M representing the distance between both fists wrapped around the back by a predetermined calculation method using the processing results of steps S403 to S406 for the target image, which is the image of the evaluation target frame.

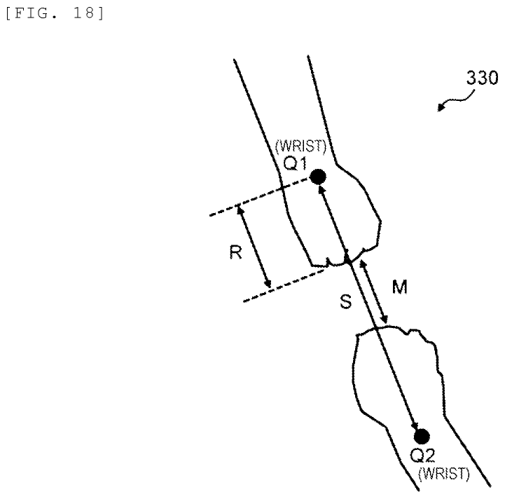

FIG. 18 is a diagram for explaining a method for calculating the number of pixels M between fists. FIG. 18 shows an image 330 near both fists in the image of an evaluation target frame when the subject 2 has their arms behind the back. A specific method of calculating the number of pixels M between the fists in step S408 will be described with reference to the image 330 of FIG. 18.

In calculating the number of fist-width pixels M, the image processing unit 111 first calculates the number of pixels between the point Q1, which is the joint position of the left wrist, and the point Q2, which is the joint position of the right wrist, from the image 330, thereby obtaining the number of pixels S between both wrists.

Next, the image processing unit 111 calculates the number of pixels M between the fists by subtracting from the number of pixels S between the wrists the value obtained by doubling the number of pixels R between the first widths calculated in step S406. That is, the number of pixels M between fists is calculated by “S−R x 2”.

Returning to the description of FIG. 14, after calculating the number of pixels between the fists M in step S408, the image processing unit 111 calculates an evaluation score (step S409). The evaluation score is calculated based on the number of fist-width pixels R calculated in step S406 and the number of pixels M between fists calculated in step S408, specifically, the value that is not greater than “1-M/R” and “0” is used as the evaluation score.

Then, the image processing unit 111 records the evaluation score calculated in step S409 in the evaluation score management table 122 (step S410) and returns to step S401 for processing the image of the next frame.

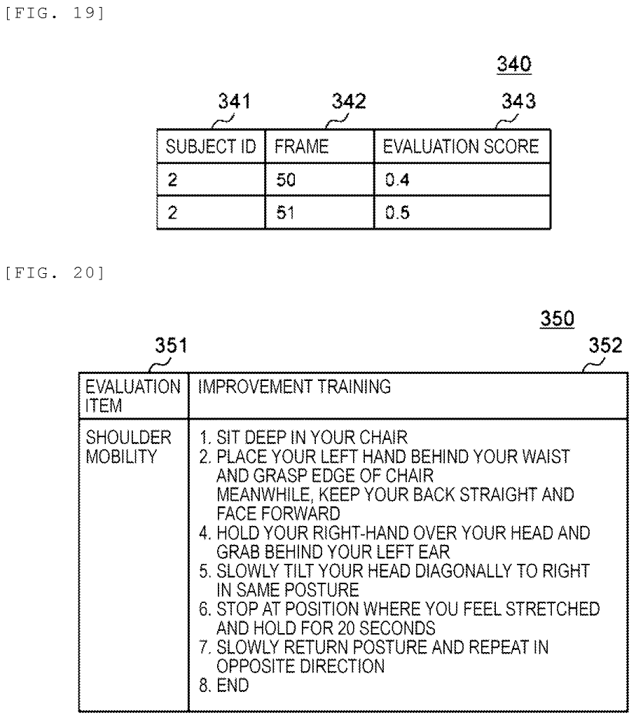

FIG. 19 is a diagram illustrating an example of the evaluation score management table 122. The evaluation score management table 340 shown in FIG. 19 is an example of the evaluation score management table 122 in Example 2 and is table data configured to include items of subject ID 341, frame 342, and evaluation score 343. Since the configuration of the evaluation score management table 340 is the same as the evaluation score management table 260 shown in FIG. 11 in Example 1, the detailed description thereof will be omitted. However, in the evaluation score 343, the evaluation score calculated in step S409 is recorded.

As described above, by performing the processing of steps S401 to S410 in FIG. 14, the image processing unit 111 can calculate the evaluation score from the still image of the evaluation target frame included the measurement video received from the measurement device 11 and can record the score in the evaluation score management table 122.

Next, physical ability evaluation processing in Example 2 will be described with reference to FIG. 15. As in Example 1, the physical ability evaluation processing is executed by the physical ability evaluation unit 112 for the still images of each frame included in the measurement video received from the measuring device 11 by measuring the physical information of the subject 2 after the image processing unit 111 has completed the evaluation score calculation processing shown in FIG. 14.

According to FIG. 15, the physical ability evaluation section 112 refers to the joint position estimation result table 310 shown in FIG. 16, searches for the record with the shortest distance between both fists among the records with frames 212 corresponding to an evaluation target frame, and obtains the frame 312 of that record (step S501). In the present example, the 50th and 51st frames are the evaluation target frames. Regarding the search for the record with the shortest distance between the two fists, assuming that the size of the first is constant, the distance between both fists can also be replaced with the distance between the wrists, the distance between the xy coordinates of the right wrist (x coordinate 313 of the right wrist, y coordinate of the right wrist 314) and the xy coordinates of the left wrist (x coordinate 315 of the left wrist, y coordinate 316 of the left wrist) is calculated to search for the record indicating the shortest distance. Alternatively, the number of pixels M between fists obtained in step S408 of the evaluation score calculation processing shown in FIG. 14 may be stored separately, and a record indicating the shortest distance may be retrieved based on the number of pixels M between fists. In the present example, the distance between both fists at the 51st frame is the shortest frame.

Next, the physical ability evaluation unit 112 refers to the evaluation score management table 340 illustrated in FIG. 19, acquires the evaluation score of the frame acquired in step S501, and stores the acquired evaluation score in a predetermined storage unit (for example, the hard disk 103) as the flexibility evaluation result of shoulder joint of the subject 2 (shoulder joint flexibility evaluation result) (step S502). Specifically, since the frame acquired in step S501 is “51”, referring to the evaluation score management table 340, the evaluation score “0.5” of the 51st frame is stored as the shoulder joint flexibility evaluation result.

As described above, by performing the processing of steps S501 and S502 in FIG. 15, the physical ability evaluation unit 112 can evaluate the evaluation score calculated from the measurement video by the evaluation score calculation processing and determine the physical ability evaluation result (specifically, the flexibility evaluation result of the shoulder joint of the subject 2).

Then, as in Example 1, after the physical ability evaluation processing is completed, the processing of steps S105 to S112 in FIG. 3 are performed. When the personnel and general affairs terminal 21 or the subject terminal 22 requests the provision of evaluation results, the evaluation result notification unit 113 creates and provides an evaluation report.

Regarding the method of creating an evaluation report in Example 2, although the specific content of the physical ability evaluation (evaluation of ankle flexibility or evaluation of shoulder joint flexibility) is different, the preparation procedure other than that is the same as in Example 1. Therefore, only specific examples of the improvement training management table 124 and the evaluation report in Example 2 will be shown below, and the detailed description thereof will be omitted.

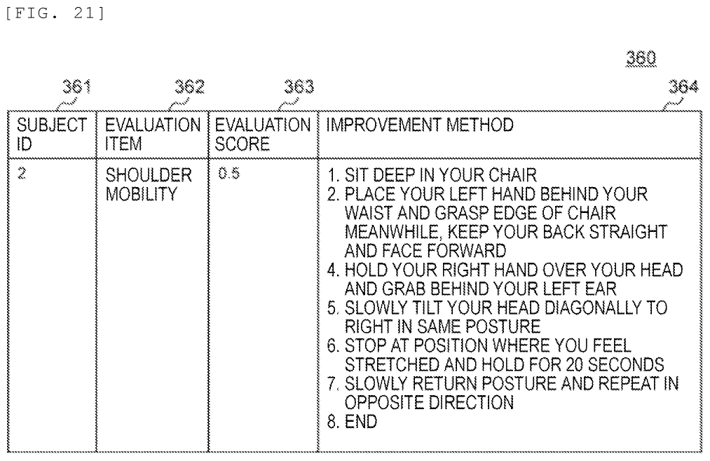

FIG. 20 is a diagram illustrating an example of the improvement training management table 124. The improvement training management table 350 shown in FIG. 20 is an example of the improvement training management table 124 in Example 2 and is table data configured to include items of evaluation item 351 and improvement training 352.

FIG. 21 is a diagram illustrating an example of an evaluation report. The evaluation report 360 shown in FIG. 21 is an example of an evaluation report regarding the physical ability evaluation of the shoulder joint flexibility of the subject 2, and is table data configured to include items of subject ID 361, evaluation item 362, evaluation score 363, and improvement method 364.

As described above, the evaluation result notification unit 113 can create an evaluation report 360 including the evaluation result of the physical ability (shoulder joint flexibility) evaluated from the measurement result of the subject 2 and the improvement training method based on the evaluation result and can provide the created evaluation report 360 to the personnel and general affairs terminal 21 and the subject terminal 22.

As described above, in Example 2, the physical ability evaluation system 1 (physical ability evaluation server 10) can evaluate the physical ability by using an inexpensive device such as a camera 14 capable of capturing videos instead of an expensive device such as a wearable device, based on the captured measurement video of the movement and posture of the subject 2 without the need for time-consuming preparation such as attachment of the sensor. Therefore, it is possible to reduce the time and cost required for physical ability evaluation.

In the physical ability evaluation system 1 (physical ability evaluation server 10) according to Example 2, based on the play timing (playing position) of the model action video played by the model action video player 12, by managing the frames of the movements and postures performed by the subject 2 in the measurement video, the timing to acquire the physique information of the subject 2 (physical information acquisition target frame) and the timing to evaluate physical ability (evaluation target frame) in the measurement video can be specified. The physical ability evaluation system 1 (physical ability evaluation server 10) acquires the physical information of the subject 2 by physique estimation, acquires the human area coordinates by segmentation, and uses the acquisition results, thereby it is possible to acquire the feature amount to be evaluated (the number of pixels M between fists representing the distance between the fists turned to the back) with high accuracy. Since the physical ability evaluation system 1 (physical ability evaluation server 10) evaluates the physical ability based on the feature amount of the evaluation target acquired as described above, the automatic evaluation of physical ability can be performed based on the highly accurate extraction of the human area.

In Example 2, the physical ability evaluation system 1 (physical ability evaluation server 10) can provide not only the result of the automatic evaluation of physical ability but also the improvement method based on the result by presenting the evaluation report. The person in charge of human resources can optimize the assigned department of the subject 2, and the subject 2 can learn the training for improving their own physical ability. Thus, according to the physical ability evaluation system 1 (physical ability evaluation server 10), by optimizing assigned departments and proposing improvement training based on the physical work ability evaluation results, productivity improvement and working life extension of middle-aged and older workers can be achieved.

Note that the present invention is not limited to the above-described embodiments and examples and includes various modifications. For example, the above-described embodiments have been described in detail in order to explain the present invention in an easy-to-understand manner and are not necessarily limited to those having all the described configurations. It is possible to add, delete, or replace part of the configuration of the embodiment or example with another configuration.

Each of the above configurations, functions, processing units, processing means, and the like may be implemented in hardware, for example, by designing a part or all of them with an integrated circuit. Each of the above configurations, functions, and the like may be implemented by software by a processor interpreting and executing a program for implementing each function. Information such as programs, tables, and files that implement each function can be stored in recording devices such as memories, hard disks, solid state drives (SSDs), or recording media such as IC cards, SD cards, and DVDs.

The control lines and information lines in the drawings show what is considered necessary for explanation, and not all control lines and information lines are necessarily shown on the product. In reality, it may be considered that almost all configurations are interconnected.

### REFERENCE SIGNS LIST

1: physical ability evaluation system
2: subject
10: physical ability evaluation server
11: measuring device
12: model action video player
13: internet
14: camera
15: projector
16: screen
17: yoga mat
21: personnel and general affairs terminal
22: subject terminal
101: CPU
102: memory
103: hard disk
104: communication interface
111: image processing unit
112: physical ability evaluation unit
113: evaluation result notification unit
121, 210, 310: joint position estimation result table
122, 260, 340: evaluation score management table
123, 230: evaluation parameter management table
124, 270, 350: improvement training management table
220, 320: human area extraction result
240: model action terminal
250, 330: image
280, 360: evaluation report

1. A physical ability evaluation server that evaluates the physical ability of a subject based on a measurement video in which the subject performs a predetermined action required for evaluating the physical ability, the physical ability evaluation server comprising:
an image processing unit that that calculates the evaluation score of the physical ability by executing evaluation score calculation processing on a plurality of still images included in the measurement video;
a physical ability evaluation unit that evaluates the physical ability based on the evaluation score calculated by the image processing unit; and
an evaluation result notification unit that creates and outputs an evaluation report based on the results of the evaluation by the physical ability evaluation unit, wherein
the evaluation score calculation processing includes
a first process of acquiring joint position coordinates of the subject by physique estimation for each of a plurality of still images,
a second process of acquiring human area coordinates forming a human area of the subject by segmentation and acquiring predetermined physique information about the subject based on the human area coordinates for a first still image corresponding to a first target period among the plurality of still images, and
a third process of calculating the evaluation score of the physical ability by a predetermined calculation formula using the information acquired in the first process and the second process with respect to a second sill image corresponding to a second target period different from the first target period among the plurality of still images.

2. The physical ability evaluation server according to claim 1, wherein
in the evaluation score calculation processing, the image processing unit determines whether the still image corresponds to the first target period or the second target period based on the result of the physique estimation by the first process on the still image.

3. The physical ability evaluation server according to claim 1, wherein
when the evaluation item of physical ability is ankle flexibility,
the image processing unit sets the period in which the subject is in a standing posture as the first target period, and the period in which the subject is squatting as the second target period,
in the second process, height and sole length are calculated based on the human area coordinates obtained from the subject in a standing posture by segmentation, and
in the third process, by using the height and sole length calculated in the second process and the result of physique estimation in the first process, the inclination representing the degree of floating of the heel of the subject who is squatting is calculated, and the evaluation score relating to flexibility of the ankle is calculated based on the calculated inclination.

4. The physical ability evaluation server according to claim 1, wherein
when the physical ability evaluation item is shoulder joint flexibility,
the image processing unit sets the period in which the subject is in a standing posture as the first target period, and the period in which the subject has both arms are wrapped around their back as the second target period,
in the second process, the length from the wrist to the tip of the first is calculated based on the human area coordinates obtained from the subject in a standing posture by segmentation, and
in the third process, by using the length from the wrist to the tip of the first calculated in the second process and the result of physique estimation in the first process, the distance between both fists of the subject, the subject having both arms wrapping around their back is calculated, and the evaluation score regarding flexibility of the shoulder joint is calculated based on the calculated distance.

5. The physical ability evaluation server according to claim 1, wherein
the physical ability evaluation unit selects one evaluation score from among the evaluation scores calculated from the one or more second still images by the image processing unit, based on specific joint position coordinates acquired in the first process for the second still image, and uses the selected evaluation score as the evaluation result of the physical ability.

6. The physical ability evaluation server according to claim 1, wherein
the evaluation report includes at least the result of evaluation by the physical ability evaluation unit and improvement training recommended according to the result of the evaluation.

7. A physical ability evaluation system for evaluating the physical ability of a subject based on a measurement video in which the subject performs a predetermined action required for evaluating the physical ability, the physical ability evaluation system comprising:
a model action video player that plays a model action video for instructing the subject to execute the predetermined action;
a measuring device that controls the playing of the model action video by the model action video player and acquires the measurement video in which the subject has been photographed while playing the model action video; and
a physical ability evaluation server that evaluates the physical ability of the subject based on the measurement video received from the measuring device, wherein
the physical ability evaluation server includes
an image processing unit that calculates an evaluation score of the physical ability by executing evaluation score calculation processing on a plurality of still images included in the measurement video,
a physical ability evaluation unit that evaluates the physical ability based on the evaluation score calculated by the image processing unit, and
an evaluation result notification unit that creates and outputs an evaluation report based on the evaluation result by the physical ability evaluation unit, and
the evaluation score calculation processing includes
a first process of acquiring the joint position coordinates of the subject by physique estimation for each of the plurality of still images,
a second process of acquiring human area coordinates forming a human area of the subject by segmentation and acquiring predetermined physique information about the subject based on the human area coordinates for a first still image corresponding to a first target period among the plurality of still images, and
a third process of calculating an evaluation score of the physical ability by a predetermined calculation formula using the information acquired in the first process and the second process for a second still image corresponding to a second target period different from the first target period among the plurality of still images.

8. The physical ability evaluation system according to claim 7, wherein
the physical ability evaluation server manages the plurality of still images included in the measurement video in association with the playback position of the model action video.

9. The physical ability evaluation system according to claim 7, wherein
in the evaluation score calculation processing, the image processing unit determines whether the still image corresponds to the first target period or the second target period based on the result of physique estimation on the still image by the first process.

10. The physical ability evaluation system according to claim 8, wherein
in the evaluation score calculation processing, the image processing unit determines whether the still image corresponds to the first target period or the second target period based on the result of physique estimation on the still image by the first process and the playing position of the model action video corresponding to the still image.

11. A physical ability evaluation method with a physical ability evaluation server for evaluating the physical ability of a subject based on a measurement video in which the subject performs a predetermined action required for evaluating the physical ability, the physical ability evaluation method comprising:
an image processing step of calculating an evaluation score of the physical ability by executing an evaluation score calculation processing on a plurality of still images included in the measurement video;
a physical ability evaluation step of evaluating the physical ability based on the evaluation score calculated in the image processing step; and
an evaluation result notification step of creating and outputting an evaluation report based on the evaluation result of the physical ability evaluation step, wherein
the evaluation score calculation processing in the image processing step includes
a first process of acquiring the joint position coordinates of the subject by physique estimation for each of the plurality of still images,
a second process of acquiring human area coordinates forming a human area of the subject by segmentation and acquiring predetermined physique information about the subject based on the human area coordinates for a first still image corresponding to a first target period among the plurality of still images, and
a third process of calculating an evaluation score of the physical ability by a predetermined calculation formula using the information acquired in the first process and the second process for a second still image corresponding to a second target period different from the first target period among the plurality of still images.
